(19) United States
(12) Patent Application Publication
(10) Pub. No.: US 2025/0284593 A1
(43) Pub. Date: Sep. 11, 2025

(54) INFORMATION PROCESSING SYSTEM

(71) Applicant: TOHOKU UNIVERSITY, Sendai-shi, Miyagi (JP)

(72) Inventor: Susumu FUJII, Sendai-shi (JP)

(21) Appl. No.: 18/860,067

(22) PCT Filed: Mar. 10, 2023
(86) PCT No.: PCT/JP2023/009307

§ 371 Date: Oct. 25, 2024

(30) Foreign Application Priority Data
Apr. 28, 2022 (JP) 2022-074989

(51) Int. Cl. G06F 11/14

(52) U.S. Cl. CPC G06F 11/1448

(57) ABSTRACT

An information processing system includes a snapshot information acquisition unit configured to acquire snapshot information in which unit meaning information and situation information are associated with each other, the unit meaning information being obtained by dividing information indicating a state of a subject into a meaningful unit, the situation information including at least timing information indicating a timing at which the state indicated by the unit meaning information occurs and identification information for identifying the subject; and a process information generation unit configured to generate process information in which the snapshot information is arranged in a time series for each of the subjects based on the timing information.

### TECHNICAL FIELD

The present invention information processing system.

### BACKGROUND ART

In the related art, a technique for generating a database for work support based on a definition regarding a meaning of information by a human recognition is disclosed (for example, refer to Patent Literature 1).

### CITATION LIST

### Patent Literature

Patent Literature 1: JP2020-140604A.

### SUMMARY OF INVENTION

### Technical Problem

However, in the technique in the related art as described above, in a case where the database is generated based on the definition by the human recognition, there is a problem in that work of imparting the human recognition to information is necessary, and an enormous number of man-hours are required.

The present invention has been made to solve the above problem, and an object of the present invention is to provide an information processing system capable of generating structured information based on an enormous amount of snapshot information to improve accuracy of the structured information.

### Solution to Problem

An embodiment of the present invention relates to an information processing system including a snapshot information acquisition unit configured to acquire snapshot information in which unit meaning information and situation information are associated with each other, the unit meaning information being obtained by dividing information indicating a state of a subject into a meaningful unit, the situation information including at least timing information indicating a timing at which the state indicated by the unit meaning information occurs and identification information for identifying the subject; and a process information generation unit configured to generate process information in which the snapshot information is arranged in a time series for each of the subjects based on the timing information.

The above information processing system according to the embodiment of the present invention further includes a peculiarity determination unit configured to determine a peculiarity [of a part of a time series] of the process information generated by the process information generation unit based on the process information and reference information indicating a predetermined reference; a state information generation unit configured to classify the process information for groups of the peculiarity determined by the peculiarity determination unit to generate state information for each of the groups; and a structured information generation unit configured to generate structured information by combining the state information in different groups in the generated state information.

The above information processing system according to the embodiment of the present invention further includes a meaning imparting unit configured to impart a meaning to the state indicated by the structured information.

The above information processing system according to the embodiment of the present invention further includes a communication information acquisition unit configured to acquire communication information indicating a content of communication between users; an extraction unit configured to extract, based on the acquired communication information and the structured information, information, which is required to be complemented based on the structured information, in a time series of the communication as complementary information; and a presentation unit configured to present the extracted complementary information to the users.

The above information processing system according to the embodiment of the present invention further includes an information supply t configured to supply, to the snapshot information acquisition unit, information indicating a state of a subject obtained as a result of the presentation of the complementary information presented by the presentation unit.

An embodiment of the present invention relates to an information processing system including: a process information acquisition unit configured to acquire process information in which snapshot information, in which unit meaning information obtained by dividing information indicating a state of a subject into a meaningful unit and situation information are associated with each other, is arranged in a time series for each of the subjects based on timing information indicating a timing at which the state indicated by the unit meaning information occurs, the situation information including at least timing information and identification information for identifying the subject; a peculiarity determination unit configured to determine a peculiarity of the acquired process information based on the process information and reference information indicating a predetermined reference; a state information generation unit configured to classify the process information for groups of the peculiarity determined by the peculiarity determination unit to generate state information for each of the groups; and a structured information generation unit configured to generate structured information by combining the state information in different groups in the generated state information.

### Advantageous Effects of Invention

According to the present invention, it is possible to generate structured information based on an enormous amount of snapshot information to improve accuracy of the structured information.

### BRIEF DESCRIPTION OF DRAWINGS

FIG. 1 is a diagram showing an example of a configuration of an information processing system according to an embodiment.

FIG. 2 is a diagram showing an example of a processing flow in a recognition mode of the information processing system according to the present embodiment.

FIG. 3 is a diagram showing an example of a structure of snapshot information according to the present embodiment.

FIG. 4 is a diagram showing an example of a structure of process information according to the present embodiment.

FIG. 5 is a diagram showing an example of a structure of state information according to the present embodiment.

FIG. 6 is a diagram showing an example of the structure of structured information according to the present embodiment.

FIG. 7 is a diagram showing an example of a structure of meaning matching structured information according to the present embodiment.

FIG. 8 is a diagram showing an example of a configuration of the information processing system in an application mode according to the present embodiment.

FIG. 9 is a diagram showing an example of a processing flow in the application mode according to the present embodiment.

### DESCRIPTION OF EMBODIMENTS

### Overview of Information Processing System 1

An information processing system 1 of the present embodiment converts human thought into data in a computer-recognizable form, classifies a thought process (process), and accumulates the classified thought process in a database. The information processing system 1 supports human thought by complementing the human thought process with information accumulated in the database.

The information processing system 1 can be used in various fields. In the present embodiment, a case in which the information processing is system 1 used as s an examination navigation system at the time of examination of a patient by a doctor is described as an example. In this example, a user of the information processing system 1 includes a doctor and a patient.

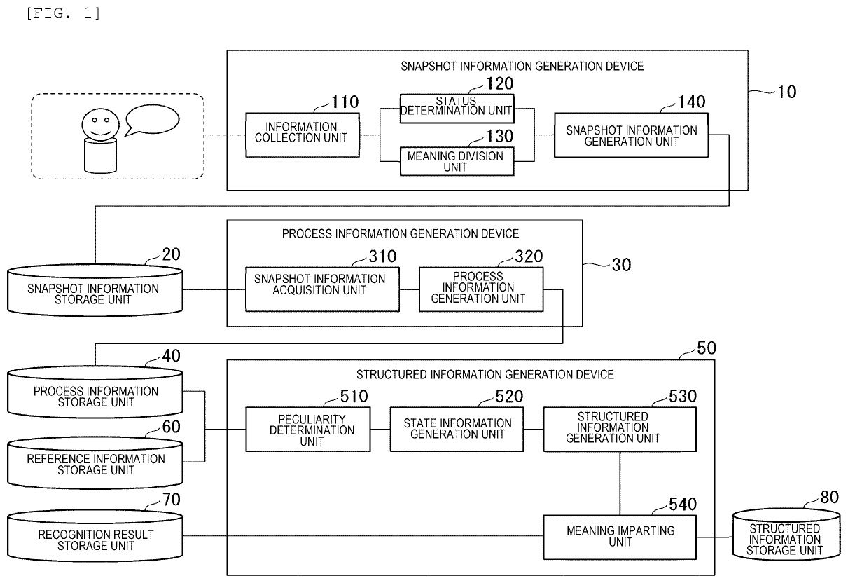

Hereinafter, the following description is divided into a process (recognition mode) in which a computer of the information processing system 1 performs recognition and a process (application mode) in which the computer applies a recognition result. First, a configuration of the information processing system 1 in the recognition mode will be described with reference to FIG. 1.

The recognition mode may be referred to as a stage (learning stage) of accumulating information in a database. The application mode may be referred to as a stage (use stage) of using information accumulated in the database.

### Configuration of Information Processing System 1 in Recognition Mode

FIG. 1 is a diagram showing an example of the configuration of the information processing system 1 according to the present embodiment.

The information processing system 1 includes a snapshot information generation device 10, a process information generation device 30, and a structured information generation device 50.

Each of the devices includes an arithmetic circuit such as a central processing unit (CPU) and a storage device (both not shown), and operates according to a program built in the storage device (or a program supplied from the outside). Each of the devices provides various functions in cooperation with the arithmetic circuit and the storage device. Among the functions provided by the devices, a functional unit provided according to a software configuration is referred to as a software functional unit, and a functional unit provided according to a hardware configuration is also referred to as a hardware functional unit. In the following description, the software functional unit and the hardware functional unit are simply referred to as a functional unit.

The snapshot information generation device 10, the process information generation device 30, and the structured information generation device 50 may be integrated and implemented in a single computer device or may be divided and implemented in a plurality of computer devices. The snapshot information generation device 10, the process information generation device 30, and the structured information generation device 50 may be implemented in a virtual device such as a cloud server.

The information processing system 1 includes a snapshot information storage unit 20, a process information storage unit 40, a reference information storage unit 60, a recognition result storage unit 70, and a structured information storage unit 80. Each of the storage units may be implemented by a storage device included in any one of the snapshot information generation device 10, the process information generation device 30, and the structured information generation device 50. Each of the storage units may be implemented by a virtual device such as a cloud server.

### Functional Configuration of Snapshot Information Generation Device 10

The snapshot information generation device 10 includes an information collection unit 110, a status determination unit 120, a meaning division unit 130, and a snapshot information generation unit 140 as functional units.

The information collection unit 110 collects information indicating a state of an information collection subject. The information indicating the state of the information collection subject (hereinafter, also simply referred to as a subject) broadly includes information related to the state of the subject, such as a conversation, an utterance, a post on a social networking service (SNS), an electronic mail, a meeting minutes, health information obtained from wearable devices, purchasing history of goods and food, results of health checkups and medical tests of the subject, location information of the subject, a scattering state of fine particles such as pollen and air pollutants, and temperature, humidity, and air pressure conditions.

The information collection unit 110 may include a sound collection unit (microphone) that acquires audio information, an optical character reading unit (OCR) that acquires text information from paper media, a communication unit or storage medium reading unit that acquires text or audio information from other computer devices, or the like.

In addition to a content of the collected information (What), the information collection unit 110 also collects the information (so-called 5W1H) if there is information that identifies an individual sender (Who), and indicates a date and time when the information is sent (When), a place (Where), a situation (How), a reason for sending the information (Why), and the like.

Note that any one of the pieces of information of 5W1H may be missing (for example, some of information may be a Null value). For example, when the information to be collected is weather conditions around the subject, there is no reason (Why) for the information (weather conditions) to occur. In this example, the information collection unit 110 collects information related to weather conditions in which the reason (Why) is missing among 5W1H.

The information collection unit 110 outputs the collected information to the status determination unit 120 and the meaning division unit 130.

The status determination unit 120 determines a status in which the information collected by the information collection unit 110 is transmitted. If the information collected by the information collection unit 110 includes the information of 5W1H described above, the status determination unit 120 determines the status based on the information of 5W1H. For example, the information collected by the information collection unit 110 may include information (timing information) indicating a timing at which the state occurs and information (identification information) for identifying the subject. In this case, the status determination unit 120 determines that the information collected by the information collection unit 110 is information issued by the subject indicated by the identification information at the timing indicated by the timing information.

In the following description, the timing information and the identification information are also collectively referred to as situation information.

The status determination unit 120 outputs the situation information that is the determination result to the snapshot information generation unit 140.

The meaning division unit 130 divides the information collected by the information collection unit 110 into units that have meaning in terms of content. The information divided by the meaning division unit 130 is also referred to as unit meaning information. For example, the meaning division unit 130 divides the information into unit meaning information by well-known natural language processing and automatic structuring. In the following description, the unit meaning information is also referred to as atom. The unit meaning information (atom) may be information divided into the smallest unit that has meaning in natural language.

The meaning division unit 130 outputs the unit meaning information to the snapshot information generation unit 140.

The snapshot information generation unit 140 generates a snapshot information SSD by associating the situation information output by the status determination unit 120 with the unit meaning information output by the meaning division unit 130.

The snapshot information generation unit 140 causes the snapshot information storage unit 20 to store the generated snapshot information SSD.

### Functional Configuration of Process Information Generation Device 30

The process information generation device 30 includes a snapshot information acquisition unit 310 and a process information generation unit 320 as functional units thereof.

The snapshot information acquisition unit 310 acquires the snapshot information SSD from the snapshot information storage unit 20. As described above, the snapshot information SSD is information in which the timing information and the situation information are associated with each other.

That is, the snapshot information acquisition unit 310 acquires the snapshot information SSD in which the unit meaning information and the situation information are associated with each other, here, the unit meaning information is obtained by dividing the information indicating the state of the subject into meaningful units, and the situation information includes at least the timing information indicating the timing at which the state indicated by the unit meaning information occurs and the identification information for identifying the subject.

Note that the information indicating the state of the subject described here includes information indicating the state of the subject himself or herself and information indicating a condition of an environment where the subject is located (for example, the weather and air pressure in a place where the subject is located). The environment where the subject is located does not matter directly or indirectly.

The snapshot information acquisition unit 310 outputs the acquired snapshot information SSD to the process information generation unit 320.

The process information generation unit 320 generates process information PD in which the snapshot information SSD is arranged in a time series for each of the subjects based on the timing information.

The process information generation unit 320 causes the process information storage unit 40 to store the generated process information PD.

### Functional Configuration of Structured Information Generation Device 50

The structured information generation device 50 includes a peculiarity determination unit 510, a state information generation unit 520, a structured information generation unit 530, and a meaning imparting unit 540 as functional units thereof.

The peculiarity determination unit 510 acquires the process information PD stored in the process information storage unit 40 and reference information stored in the reference information storage unit 60. That is, the peculiarity determination unit 510 functions as a process information acquisition unit that acquires the process information PD from the process information storage unit 40.

The peculiarity determination unit 510 determines a peculiarity of the process information PD based on the process information PD and the reference information.

Here, the reference information is information indicating a standard (or reference) status among statuses indicated by the process information PD. The reference information is also referred to as master condition data.

The peculiarity determination unit 510 compares the process information PD stored in the process information storage unit 40 with the reference information, and determines whether the status indicated by the process information PD deviates from the standard status (or the reference status). When it is determined that the status indicated by the process information PD deviates from the standard status (or the reference status), the peculiarity determination unit 510 determines that the process information PD has a peculiarity.

The peculiarity determination unit 510 may determine the peculiarity of a part of the time series among the time series of the process information PD.

The peculiarity determination unit 510 determines a type of the peculiarity of the process information PD and groups the process information PD for each of the types of the peculiarity.

The state information generation unit 520 classifies the process information PD for the groups of the peculiarity determined by the peculiarity determination unit 510 to generate the state information CD for each of the groups.

The structured information generation unit 530 generates structured information MS by combining state information CD in different groups in the generated state information CD.

The meaning imparting unit 540 imparts a human-recognizable meaning to the state indicated by the structured information MS. Specifically, the meaning imparting unit 540 acquires a human recognition result stored in the recognition result storage unit 70. The meaning imparting unit 540 compares the acquired recognition result with the state indicated by the structured information MS, and thereby imparting a human-recognizable meaning to the state indicated by the structured information MS.

Note that imparting a human-recognizable meaning to the state indicated by the structured information MS is also referred to as matching the structured information MS with the meaning. Information in which the human-recognizable meaning is imparted to the state indicated by the structured information MS is also referred to as a meaning matching structured information IMS. The meaning matching structured information IMS is also referred to as “identify-MAP (iMAP)”.

The meaning imparting unit 540 causes the structured information storage unit 80 to store the meaning matching structured information IMS.

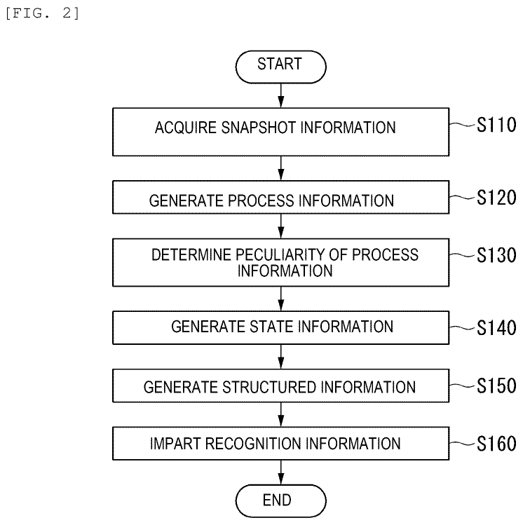

Next, a processing flow in which the process information generation device 30 and the structured information generation device 50 generate meaning matching structured information IMS based on the snapshot information SSD generated by the snapshot information generation device 10 will be described with reference to FIG. 2.

### Processing Flow in Recognition Mode

FIG. 2 is a diagram showing an example of a processing flow in the recognition mode of the information processing system 1 according to the present embodiment.

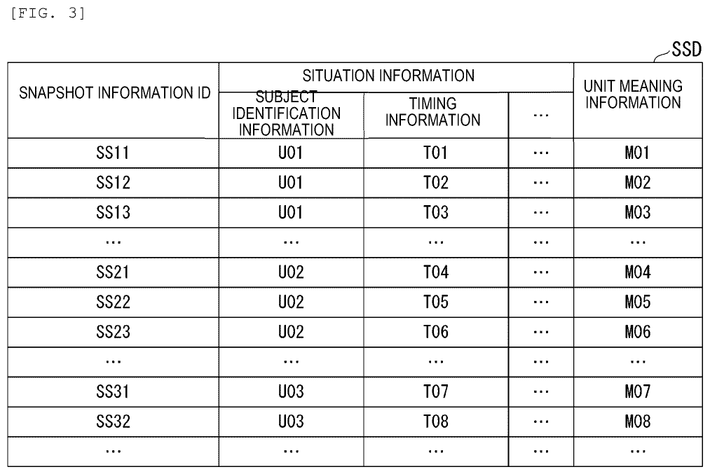

The snapshot information acquisition unit 310 acquires the snapshot information SSD from the snapshot information storage unit 20 (step S110). An example of the snapshot information SSD will be described with reference to FIG. 3.

### Example of Snapshot Information

FIG. 3 is a diagram showing an example of a structure of the snapshot information SSD according to the present embodiment. In the snapshot information SSD, a snapshot information ID, situation information, and unit meaning information are associated with each other.

The snapshot information SSD is divided for each of the subjects and at each of timings, and stored in the snapshot information storage unit 20.

For example, each of snapshot information IDs (SS11 to SS13) is the snapshot information SSD related to a subject U01. Each of snapshot information IDs (SS21 to SS23) is snapshot information SSD related to a subject U02. Similarly, each of snapshot information IDs (SS31 to SS32) is snapshot information SSD related to a subject U03.

In the snapshot information SSD related to the subject U01, the snapshot information ID (SS11) indicates unit meaning information M01 which is a status generated at a timing T01.

Returning to FIG. 2, the snapshot information acquisition unit 310 outputs the acquired snapshot information SSD to the process information generation unit 320.

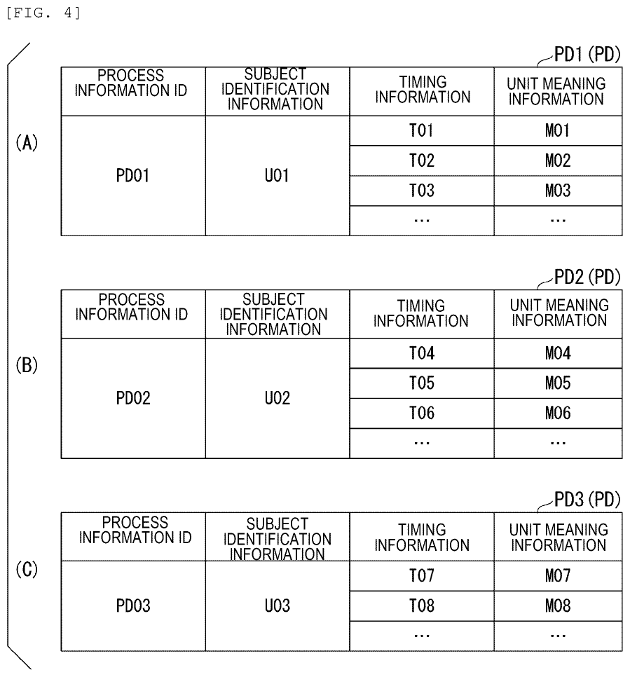

The process information generation unit 320 generates the process information PD based on the snapshot information SSD acquired in step S110 (step S120). An example of the process information PD will be described with reference to FIG. 4.

### Example of Process Information

FIG. 4 is a diagram showing an example of a structure of the process information PD according to the present embodiment. The process information PD is information in which the snapshot information SSD is arranged in a time series for each of the subjects based on the timing information. (A) of FIG. 4 shows the process information PD of the subject U01. (B) of FIG. 4 shows the process information PD of the subject U02. (C) of FIG. 4 shows the process information PD of the subject U03.

More specifically, the process information generation unit 320 extracts the snapshot information SSD of a specific subject (for example, the subject U01) from the snapshot information SSD. The process information generation unit 320 arranges the extracted snapshot information SSD of the subject U01 in a time series based on an occurrence timing indicated by the timing information. The process information generation unit 320 generates the snapshot information SSD of the subject U01 arranged in a time series as the process information PD of the subject U01.

The process information generation unit 320 similarly generates the process information PD for other subjects (for example, the subject U02, the subject U03, . . . ) by arranging the snapshot information SSD in a time series.

The process information generation unit 320 causes the process information storage unit 40 to store the generated process information PD of each of the subjects.

Returning to FIG. 2, the peculiarity determination unit 510 determines the peculiarity of the process information PD (step S130).

Specifically, as described above, the peculiarity determination unit 510 compares the process information PD generated in step S120 with the pre-stored reference information to determine whether the process information PD has a peculiarity.

Here, both the reference information and the process information PD are expressed in a format that can be compared by a computer (for example, a binarized data group). Therefore, it is difficult for a human to directly understand what the contents of the reference information and the process information PD indicate (for example, the meaning of information).

That is, the peculiarity determination unit 510 directly determines the peculiarity of the process information PD which has a data format that is difficult for a human to understand directly. In other words, the peculiarity determination unit 510 determines the peculiarity of the process information PD based on a data format that can be directly compared by a computer, rather than based on a definition regarding the meaning of information by a human recognition (for example, ontology).

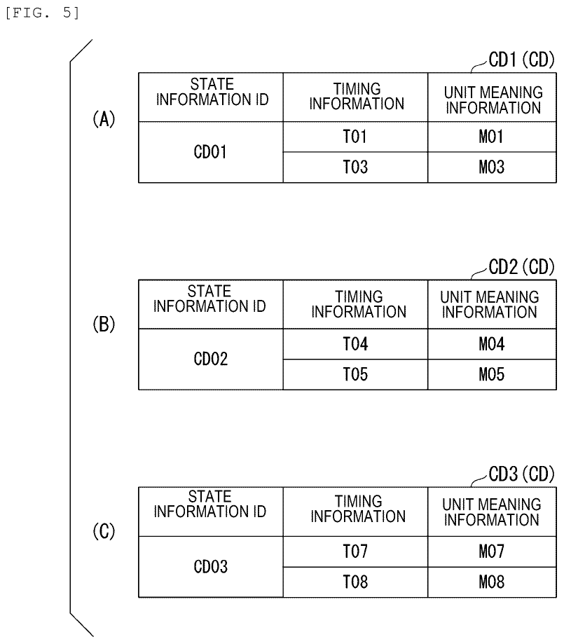

The state information generation unit 520 generates the state information CD by classifying the process information PD which is determined as information having a peculiarity in step S130 into a plurality of groups for each type of the peculiarity (step S140). Here, a symptom of a cold is taken as an example, a peculiarity group includes a group with a symptom of cough, a group with a symptom of runny nose, a group with a symptom of sore throat, and the like. In this example, the peculiarity can be rephrased as a main complaint state of the subject. An example of the state information CD will be described with reference to FIG. 5.

### Example of State Information

FIG. 5 is a diagram showing an example of a structure of the state information CD according to the present embodiment. (A) of FIG. 5 shows state information CD1 of a first group (for example, the group with the symptom of cough). (B) of FIG. 5 shows state information CD2 of a second group (for example, the group with the symptom of runny nose). (C) of FIG. 5 shows state information CD3 of a third group (for example, the group with the symptom of sore throat).

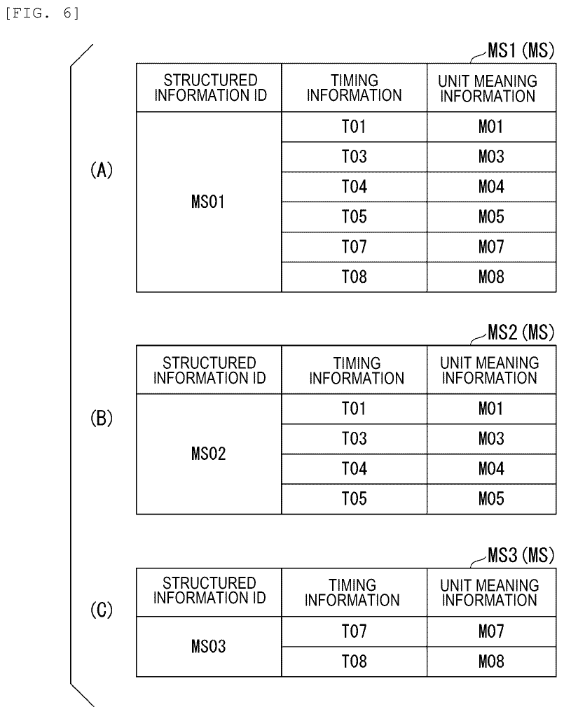

Returning to FIG. 2, the structured information generation unit 530 generates the structured information MS by combining one or more pieces of the state information CD (step S150). The structured information MS described in the present embodiment may be expressed as meta structured information or super structured information. An example of the structured information MS will be described with reference to FIG. 6.

### Example of Structured Information

FIG. 6 is a diagram showing an example of a structure of the structured information MS according to the present embodiment.

(A) of FIG. 6 shows an example of structured information MS1. The structured information MS1 is generated by combining the state information CD1, the state information CD2, and the state information CD3 in the state information CD shown in FIG. 5.

(B) of FIG. 6 shows an example of structured information MS2. The structured information MS2 is generated by combining the state information CD1 and the state information CD2 in the state information CD shown in FIG. 5.

(C) of FIG. 6 6 shows an example of structured information MS3. The structured information MS3 is generated using the state information CD3 in the state information CD shown in FIG. 5.

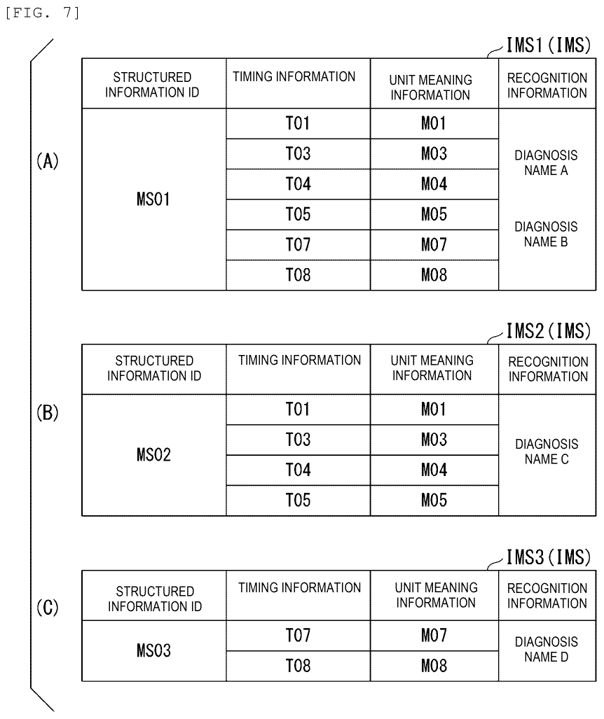

Returning to FIG. 2, the meaning imparting unit 540 generates the meaning matching structured information IMS by imparting the recognition information stored in the recognition result storage unit 70 to the structured information MS generated in step S150 (step S160). An example of the meaning matching structured information IMS will be described with reference to FIG. 7.

### Example of Meaning Matching Structured Information

FIG. 7 is a diagram showing an example of a structure of the meaning matching structured information IMS according to the present embodiment. As described above, in this example, the structured information MS1 is generated by combining the state information CD1 (group: symptom of cough), the state information CD2 (group: symptom of runny nose), and the state information CD3 (group: symptom of sore throat). In the case of the combination of symptoms, if a diagnosis name A or a diagnosis name B applies thereto, the recognition result storage unit 70 stores the recognition result based on the information to be analyzed (for example, communication information described later). In this case, the meaning imparting unit 540 imparts the diagnosis name A or the diagnosis name B to the structured information MS1.

Similarly, the meaning imparting unit 540 imparts a diagnosis name C to the structured information MS2, and imparts a diagnosis name D to the structured information MS3.

Returning to FIG. 2, the meaning imparting unit 540 causes the structured information storage unit 80 to store the meaning matching structured information IMS to which the recognition information is imparted, and ends the series of processing.

### Configuration of Information Processing System 1 in Application Mode

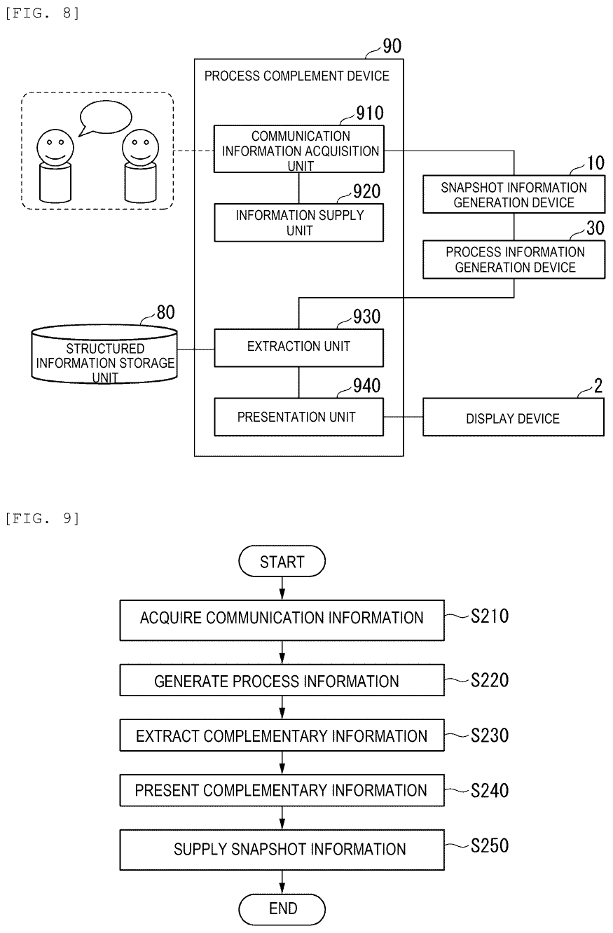

Next, the configuration of the information processing system 1 in the application mode will be described with reference to FIG. 8. The same configurations as those of the information processing system 1 described in the above recognition mode are denoted by the same reference numerals, and descriptions thereof are omitted.

FIG. 8 is a diagram showing an example of the configuration of the information processing system 1 in the application mode according to the present embodiment. In the application mode, the information processing system 1 differs from the configuration in the recognition mode in that the information processing system 1 includes a process complement device 90.

The process complement device 90 includes a communication information acquisition unit 910, an information supply unit 920, an extraction unit 930, and a presentation unit 940 as functional units thereof.

The communication information acquisition unit 910 acquires communication information indicating contents of communication between users.

The extraction unit 930 extracts, based on the acquired communication information and the structured information MS, information required to be complemented based on the structured information MS as complementary information in a time series of the communication.

The presentation unit 940 presents the extracted complementary information to the user.

The information supply unit 920 supplies information indicating the state of the subject obtained as a result of the presentation of the complementary information presented by the presentation unit 940 to the snapshot information acquisition unit 310.

A more specific processing flow in the application mode will be described with reference to FIG. 9.

### Flow in Application Mode

FIG. 9 is a diagram showing an example of a processing flow in the application mode of the present embodiment. In this example, a case where the information processing system 1 is used as an examination navigation system during the examination of a patient by a doctor will be described. In this case, the user of the information processing system 1 includes a doctor and a patient.

The communication information acquisition unit 910 acquires communication information (step S210). In this example, the communication information acquisition unit 910 includes, for example, a sound collection unit (microphone) and an information acquisition unit from an electronic medical record system. The communication information acquisition unit 910 acquires a conversation between the doctor and the patient during the examination, a description content of the electronic medical record, and the like as communication information.

The communication information acquisition unit 910 outputs the acquired communication information to the snapshot information generation device 10 (step S220). The snapshot information generation device 10 generates the snapshot information SSD based on the communication information. The snapshot information storage unit 20 generates the process information PD based on the generated snapshot information SSD. The process information PD generated in step S220 indicates a process of a content of communication between the doctor and the patient in the application mode (that is, a content exchanged in real time).

The extraction unit 930 acquires the process information PD generated by the snapshot information storage unit 20 in step S220 (that is, the process information PD generated in real time) (step S230). The extraction unit 930 compares the process information PD generated in the real time with the meaning matching structured information IMS stored in the structured information storage unit 80 (that is, the meaning matching structured information IMS generated in the recognition mode). The extraction unit 930 extracts information to be complemented based on the structured information MS as complementary information in time series of communication based on the result of the comparison.

For example, in correspondence with information to which the computer-recognizable or the human-recognizable meaning is imparted, coincidence is significantly improved if there is a blood glucose level, and in this case, if the communication information exchanged between the doctor and the patient during the examination does not include information related to the blood glucose level (inspection value), the blood glucose level (inspection value) is requested.

For example, it is assumed that the doctor and the patient during the examination have a conversation about symptoms such as “I started coughing yesterday”, “I went to sleep with coughing last night”, and “I started having a runny nose this morning”.

In this case, in step S220, the snapshot information SSD1 including the timing T01 “yesterday” and the unit meaning information M01 “I started coughing”, and the snapshot information SSD2 including the timing T03 “this morning” and the unit meaning information M03 “I started having a runny nose” are generated.

Further, in step S220, the process information PD is generated based on the snapshot information SSD1 and the snapshot information SSD2. The process information PD indicates the passage of the symptoms such as “I started coughing yesterday” (=timing T01, unit meaning information M01) and “I started having a runny nose this morning” (=timing T03, unit meaning information M03).

The extraction unit 930 searches the meaning matching structured information IMS stored in the structured information storage unit 80 using the generated process information PD as a search key. As a result of this search, for example, in the case of an example of the meaning matching structured information IMS shown in FIG. 7, “timing T01, unit meaning information M01”, and “timing T03, and unit meaning information M03” of meaning matching structured information IMS1 shown in (A) of FIG. 7 are hit.

The extraction unit 930 acquires the meaning matching structured information IMS1 as information of a complement source. The extraction unit 930 extracts, as complementary information, information associated with timings T04 to T08 following the timing T03 in the time series of communication indicated by the meaning matching structured information IMS1. For example, the extraction unit 930 extracts the diagnosis name A and the diagnosis name B shown in (A) of FIG. 7 as the complementary information.

In the case of the example described above, the “timing T01, unit meaning information M01” and the “timing T03, unit meaning information M03” of meaning matching structured information IMS2 shown in (A) of FIG. 7 are also hit as information tracing the passage of the “timing T01, unit meaning information M01” and the “timing T03, and the unit meaning information M03”.

In this case, the extraction unit 930 also acquires the meaning matching structured information IMS2 as the information of the complement source. The extraction unit 930 extracts information associated with the timing T04 to T05 following the timing T03 as the complementary information in a time series of communication indicated by the meaning matching structured information IMS2. For example, the extraction unit 930 extracts the diagnosis name C shown in (B) of FIG. 7 as the complementary information.

In the case of the example described above, it is assumed that the doctor and the patient further continue the conversation and the patient says that “My throat is starting to hurt now”. In this case, the process information PD including “timing T07, unit meaning information M07” and corresponding to “My throat is starting to hurt now” is generated. When the newly generated process information PD is added, the meaning matching structured information IMS1 is hit, but the meaning matching structured information IMS2 is not hit.

In this case, the extraction unit 930 may extract the complementary information by removing the meaning matching structured information IMS2 from candidates of the complement source and using the meaning matching structured information IMS1 as the information of the complement source. For example, the extraction unit 930 excludes the diagnosis name C shown in (B) of FIG. 7, and extracts the diagnosis name A or the diagnosis name B shown in (A) of FIG. 7 as the complementary information.

Returning to FIG. 9, the presentation unit 940 presents a user (in this case, a doctor) with the complementary information extracted by the extraction unit 930 (step S240). Specifically, the presentation unit 940 is connected to, for example, a display device 2 including a liquid crystal display. In this case, the presentation unit 940 causes the display device 2 to display the complementary information (for example, the diagnosis name A or the diagnosis name B) extracted by the extraction unit 930.

The communication information acquisition unit 910 acquires, as the communication information, information indicating the state of the subject obtained as a result of the presentation of the complementary information presented by the presentation unit 940 (for example, results of further consultation with the doctor based on the complementary information) (step S250).

When a condition for providing the communication information is satisfied, for example, in a case where a consent is obtained from a patient, the information supply unit 920 supplies the communication information to the snapshot information generation device 10 as a generation source information of new snapshot information SSD.

As a result, the snapshot information SSD stored in the snapshot information storage unit 20 can be updated with new information about the conversation between the doctor and the patient. According to such a configuration, more snapshot information SSD can be collected. According to the information processing system 1 configured in this way, accuracy of the meaning matching structured information IMS can be further improved.

As described above, the information processing system 1 according to the present embodiment includes the process information generation device 30 and the structured information generation device 50, and generates the structured information MS from the snapshot information SSD.

On the one hand, in the related art, it has been proposed to generate a database for work support based on a definition regarding a meaning of information by a human recognition. However, in a case where the database is generated based on the definition by the human recognition, there is a problem in that work of imparting the human recognition to information is necessary, and an enormous number of man-hours are required.

According to the information processing system 1 of the present embodiment, the structured information MS is generated in the data format on a computer, which is difficult for a human to directly understand, so that the structured information MS can be generated without the work of imparting the human recognition to the information.

Therefore, the information processing system 1 can generate the structured information MS based on an enormous amount of snapshot information SSD.

Therefore, according to the information processing system 1 of the present embodiment, it is also possible to generate the structured information MS by using, as the snapshot information SSD, information that a human is not aware of, or information that a human is aware of but difficult to capture because the amount of information is too large. Therefore, according to the information processing system 1 of the present embodiment, the amount and accuracy of the structured information MS can be improved, and more appropriate complementary information can be presented.

The information processing system 1 according to the present embodiment generates the meaning matching structured information IMS in which the human recognition is imparted to the structured information MS. According to the information processing system 1 configured as described above, even if the structured information MS is constructed in a data format that is difficult for a human to directly understand, information can be provided in a human-recognizable format.

Therefore, according to the information processing system 1 of the present embodiment, it is possible to simultaneously improve the amount and accuracy of the structured information MS based on an enormous amount of snapshot information SSD and present the content of the structured information MS in a human-understandable format.

Although embodiments of the present invention have been described in detail above with reference to the drawings, the specific configuration is not limited to these embodiments, and appropriate modifications can be made without departing from the gist of the present invention. The configurations described in the above embodiments may be combined.

Each of the units of each of the devices in the above embodiments may be implemented by dedicated hardware, or may be implemented by a memory and a microprocessor.

Each of the units of each of the devices may be configured by a memory and a central processing unit (CPU), and may implement a function by loading a program for implementing a function of the unit of the device into the memory and executing the program.

A program for implementing the function of each of the units of each of the devices may be recorded on a computer-readable recording medium, and the program recorded on this recording medium may be read into a computer system and executed to perform processing by each of the units of the control unit. The “computer system” herein includes an OS and hardware such as a peripheral device.

Further, the “computer system” includes a home page providing environment (or display environment) when a WWW system is used.

The “computer-readable recording medium” refers to a storage device, for example, a portable medium such as a flexible disk, a magneto-optical disk, a ROM, and a CD-ROM, and a hard disk built in the computer system. Further, the “computer-readable recording medium” includes one that dynamically holds a program for a short period of time, such as a communication line when transmitting a program via a network such as the Internet or a communication line such as a telephone line, and one that holds a program for a certain period of time, such as a volatile memory in a computer system serving as a server or a client in that case. The above program may be for implementing a part of the functions described above, and may further be capable of implementing the functions described above in combination with a program already recorded in the computer system.

### Reference Signs List

1 information processing system
10 snapshot information generation device
20 snapshot information storage unit
30 process information generation device
40 process information storage unit
50 structured information generation device
60 reference information storage unit
70 recognition result storage unit
80 structured information storage unit
90 process complement device

1. An information processing system, comprising:
a snapshot information acquisition unit configured to acquire snapshot information in which unit meaning information and situation information are associated with each other, the unit meaning information being obtained by dividing information indicating a state of a subject into a meaningful unit, the situation information including at least timing information indicating a timing at which the state indicated by the unit meaning information occurs and identification information for identifying the subject; and
a process information generation unit configured to generate process information in which the snapshot information is arranged in a time series for each of the subjects based on the timing information.

2. The information processing system according to claim 1, further comprising:
a peculiarity determination unit configured to determine a peculiarity of the process information generated by the process information generation unit based on the process information and reference information indicating a predetermined reference;
a state information generation unit configured to classify the process information for groups of the peculiarity determined by the peculiarity determination unit to generate state information for each of the groups; and
a structured information generation unit configured to generate structured information by combining the state information in different groups in the generated state information.

3. The information processing system according to claim 2, further comprising:
a meaning imparting unit configured to impart a meaning to the state indicated by the structured information.

4. The information processing system according to claim 3, further comprising:
a communication information acquisition unit configured to acquire communication information indicating a content of communication between users;
an extraction unit configured to extract, based on the acquired communication information and the structured information, information, which is required to be complemented based on the structured information, in a time series of the communication as complementary information; and
a presentation unit configured to present the extracted complementary information to the users.

5. The information processing system according to claim 4, further comprising:
an information supply unit configured to supply, to the snapshot information acquisition unit, information indicating a state of a subject obtained as a result of the presentation of the complementary information presented by the presentation unit.

6. An information processing system, comprising:
a process information acquisition unit configured to acquire process information in which snapshot information, in which unit meaning information obtained by dividing information indicating a state of a subject into a meaningful unit and situation information are associated with each other, is arranged in a time series for each of the subjects based on timing information indicating a timing at which the state indicated by the unit meaning information occurs, the situation information including at least the timing information and identification information for identifying the subject;
a peculiarity determination unit configured to determine a peculiarity of the acquired process information based on the process information and reference information indicating a predetermined reference;
a state information generation unit configured to classify the process information for groups of the peculiarity determined by the peculiarity determination unit to generate state information for each of the groups; and
a structured information generation unit configured to generate structured information by combining the state information in different groups in the generated state information.
